(19) United States
(12) Patent Application Publication
(10) Pub. No.: US 2005/0271602 A1
(43) Pub. Date: Dec. 8, 2005

(54) Method for inhibiting chemical staining of teeth

(76) Inventors: Nebojsa Milanovich, Somerset, NJ (US); Thomas Boyd, Metuchen, NJ (US); Michael Prencipe, West Windsor, NJ (US)

Correspondence Address: Harness Dickey, Suite 400, 7700 Bonhomme, St. Louis, MO, 63105, US

(21) Appl. No.: 10/859,412

(22) Filed: Jun. 2, 2004

(51) Int. Cl. A61K 7/16

(52) U.S. Cl. 424/49

(57) ABSTRACT

A method for inhibiting chemical staining of a dental surface comprises contacting the dental surface, prior to said staining, with an orally acceptable polymer or copolymer comprising a plurality of monomeric groups of formula (I) 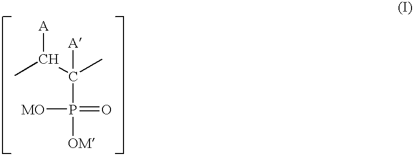
wherein (a) one of A and A′ is hydrogen and the other is a moiety (X)ₙ(R)ₘ, (b) n in individual such moieties is independently 0 or 1, (c) linking groups X if present independently comprise an oxygen, sulfur, nitrogen, phosphorus or silicon atom, (d) where n is 0, m is 1, and where n is 1, m is independently an integer from 1 to 3 as determined by X, (e) terminal groups R are independently hydrogen or C₁₋₁₈ organic radicals, and (f) M and M′ are independently selected from hydrogen, alkali metal and ammonium; said polymer or copolymer having an average molecular weight of at least about 1,000.

### FIELD

This invention relates to methods and compositions for dental care, more particularly to such methods and compositions useful for inhibiting chemical staining of dental surfaces.

### BACKGROUND

Everyday activities such as smoking or other oral use of tobacco products; eating, chewing or drinking certain foods and beverages, for example tea or coffee, or otherwise contacting teeth with staining substances such as tannins; and use of certain medicaments and oral care products such as chlorhexidine, cause undesirable discoloration of surfaces of teeth.

Typically, the problem of discoloration of dental surfaces is addressed by a remedial approach, involving for example use of abrasives, bleaching agents or coatings to remove or hide stains that have formed on such surfaces. An alternative or complementary approach involving prevention of staining has been proposed by Baig et al. (2002), J. Clin. Dent. 13(1), 19-24, who reported laboratory evaluations of reduction of stain formation by use of dentifrices comprising condensed phosphates such as sodium hexametaphosphate. It would be desirable to identify other orally acceptable agents capable of inhibiting chemical staining of teeth.

Among compounds proposed as ingredients of oral care products are phosphonic acid polymers, including for example poly(1-phosphonopropene) and poly(β-styrene phosphonic acid) as disclosed in U.S. Pat. No. 5,032,386 to Gaffar et al., such polymers being stated therein to enhance delivery of an antibacterial agent to oral surfaces.

U.S. Pat. No. 5,296,214 to Gaffar discloses polyvinylphosphonates having an average molecular weight of about 1,000 to about 1,000,000 as ingredients of oral care products said to enhance delivery of an antibacterial agent to oral surfaces.

Polyvinylphosphonates have been further disclosed as inhibitors of salivary hydrolysis of polyphosphate anticalculus agents (see, for example, U.S. Pat. No. 5,094,844 to Gaffar et al.).

Polyvinylphosphonates have been still further disclosed as anticalculus agents per se (see, for example, U.S. Pat. No. 3,429,963 to Shedlovsky).

A method of inhibiting dental plaque and gingivitis, using a composition comprising a polyvinylphosphonate having a number average molecular weight of about 4,000 to 9,100, was proposed in U.S. Pat. No. 4,816,245 to Gaffar.

It is reported in British Patent No. 1 372 199 of Colgate-Palmolive Company that polyethylene monosodium polyphosphonate having on average one phosphonate group for every 6-7 carbon atoms on the polyethylene chain “is strongly absorbed onto tooth enamel”, resulting in inhibition of bacterial adhesion and growth on treated surfaces. Other “suitable materials” are said to include “homopolymeric sodium vinyl phosphonate (M.W. 20,000)”.

U.S. Pat. No. 6,509,007 to Rajaiah et al. discloses an oral care composition comprising polybutene and one or a combination of “oral care actives” that can include an anticalculus agent, e.g., a polyvinylphosphonate, and/or a whitening agent, e.g., a peroxide.

Patents and publications cited above are incorporated herein by reference.

### SUMMARY

It has now surprisingly been discovered that certain polymers and copolymers comprising phosphonate-containing monomeric groups are effective in inhibiting formation of chemical stains on dental surfaces. Accordingly, there is now provided a method for inhibiting chemical staining of a dental surface, the method comprising contacting the dental surface, prior to exposure of the surface to a chemical staining material, with an orally acceptable polymer or copolymer that comprises a plurality of monomeric groups of formula (I) 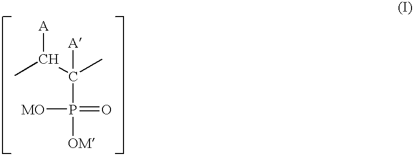
wherein:
(a) one of A and A′ is hydrogen and the other is a moiety (X)ₙ(R)ₘ,
(b) n in individual such moieties is independently 0 or 1,
(c) linking groups X if present independently comprise an oxygen, sulfur, nitrogen, phosphorus or silicon atom,
(d) where n is 0, m is 1, and where n is 1, m is independently an integer from 1 to 3 as determined by X,
(e) terminal groups R are independently hydrogen or C₁₋₁₈ organic radicals, and
(f) M and M′ are independently selected from hydrogen, alkali metal and ammonium; said polymer or copolymer having an average molecular weight of at least about 1,000.

In one embodiment of the above method, the polymer or copolymer is a polyvinylphosphonate that comprises recurring monomeric groups of formula (II) 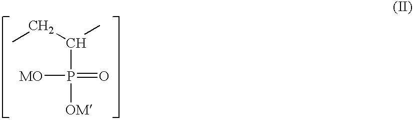
where M and M′ are each hydrogen, alkali metal or ammonium and are the same or different. Such a polyvinylphosphonate can have an average molecular weight of about 1,000 to about 100,000, in an illustrative embodiment about 22,000 to about 90,000.

In a further embodiment, there is provided an oral care composition comprising, in an orally acceptable vehicle, an amount, effective to inhibit chemical staining of a dental surface, of a polyvinylphosphonate as defined immediately above.

In methods and compositions of the invention, a whitening agent such as a peroxide is optionally present in combination with the polymer or copolymer comprising phosphonate-containing monomeric groups.

### DETAILED DESCRIPTION

A “chemical stain” herein is a discoloration of a dental surface caused by adsorption or absorption of a colored agent on or into the surface, or caused by chemical reaction of material of the dental surface (e.g., dental enamel) with a colored or noncolored agent contacting the surface. “Chemical staining” herein means formation and/or development of a chemical stain.

“Inhibition” of chemical staining as an object or result of treatment herein means reduction or prevention of stains that would otherwise form or develop subsequent to the time of the treatment. Such inhibition can range from a small but observable or measurable reduction to complete prevention of subsequent staining, by comparison with an untreated or placebo-treated dental surface.

A “dental surface” herein is a surface of a natural tooth or a hard surface of artificial dentition including a crown, cap, filling, bridge, dental implant and the like.

A step of “contacting” a dental surface with a compound or composition encompasses any procedure wherein the compound or composition comes into contact with the surface, including without limitation by rinsing (as with mouthwash), spraying (as with an oral spray), brushing (as with dentifrice), placement (as with oral strips), painting (as with liquid whitener) and chewing (as with gum).

An “orally acceptable” compound or composition is one that is not harmful to a mammal in amounts disclosed herein when retained in the mouth, without swallowing, for a period sufficient to permit effective contact with a dental surface as required herein. In general, such a compound or composition is not harmful even if unintentionally swallowed.

“Average molecular weight” herein means a weight average as opposed to a number average, except where number average molecular weight is expressly stated. Weight average molecular weight (MW_{w}) can be determined, for example, by light scattering, small angle neutron scattering (SANS) or sedimentation velocity techniques. Number average molecular weight (MWₙ) can be determined, for example, by techniques involving gel permeation chromatography, osmometry, end-group titration or colligative properties.

The present method comprises contacting a dental surface with an orally acceptable phosphonate-containing compound that is a polymer or copolymer as defined above. The method is applicable to dental surfaces of nonhuman mammals such as companion animals (e.g., dogs and cats), as well as to humans. In one embodiment the dental surface is a surface of a natural tooth of a mammal, for example a human.

Where the dental surface is substantially free of chemical stains, the present method is effective to inhibit formation and development of new chemical stains, as can occur for example by oral use of tobacco products (including smoking) or by drinking tea or coffee, subsequent to treatment according to the method. Where the dental surface already possesses some degree of chemical staining, the present method is effective to inhibit further development of the existing stain. In some embodiments, for example where the phosphonate-containing compound is present together with a dental whitening agent such as a peroxide, the present method can remove, partially or completely, an existing chemical stain as well as inhibit subsequent staining.

In one embodiment the method further comprises, after contacting the dental surface with the polymer or copolymer, exposing the dental surface to a chemical stain inducing material such as a tobacco product, tea or coffee. Chemical staining resulting from such exposure is, in this embodiment, inhibited by the prior contacting of the dental surface with the polymer or copolymer.

It is desirable that the phosphonate-containing polymer or copolymer should remain in contact with the dental surface for a period sufficient to provide effective inhibition of chemical staining. Depending on various factors including the particular phosphonate-containing compound selected, other materials optionally present in combination with the phosphonate-containing compound, the precise procedure by which contact is effected (e.g., rinsing, brushing, placement of a strip, painting or chewing) and the desired degree and/or duration of inhibition of staining, a suitable minimum period of contact can be from about 10 seconds to about 8 hours. Where the phosphonate-containing compound is applied as a component of a mouthwash, an illustrative minimum period of rinsing is about 10 seconds to about 2 minutes. Where the phosphonate-containing compound is applied as a component of a dentifrice, an illustrative minimum period of brushing is about 30 seconds to about 5 minutes, or in one embodiment at least about 1 minute, in another at least about 2 minutes. Where the phosphonate-containing compound is applied as a component of an oral strip, the strip is placed on the dental surface illustratively for a period of about 15 minutes to about 8 hours (e.g., overnight). Where the phosphonate-containing compound is applied as a component of a liquid whitener composition, the composition is painted onto the dental surface and left in place illustratively for a period of about 5 minutes to about 8 hours (e.g., overnight). Where the phosphonate-containing compound is applied as a component of a chewing gum, an illustrative minimum period of chewing is about 1 to about 20 minutes.

Increasing the degree of agitation in the mouth during rinsing, brushing or chewing can lead to improved contact of the phosphonate-containing compound with the dental surface and enhance the degree of inhibition of staining. Thus, in an embodiment where the phosphonate-containing compound is present as an ingredient of a dentifrice, vigorous brushing with the dentifrice can be particularly effective.

The phosphonate-containing compound is a polymer or copolymer comprising a plurality of monomeric groups of formula (I) above. Such polymers and copolymers are illustratively disclosed in above-cited U.S. Pat. No. 5,032,386. In one embodiment the monomeric groups are recurring groups, i.e., a plurality of similar groups are present in the polymer or copolymer. In a particular embodiment, the phosphonate-containing compound is a homopolymer.

In one embodiment, A in the monomeric groups of formula (I) is a moiety (X)ₙ(R)ₘ as hereinabove defined, and A′ is hydrogen. In another embodiment, A is hydrogen and A′ is a moiety (X)ₙ(R)ₘ as hereinabove defined. According to either one of these embodiments, (X)ₙ(R)ₘ is illustratively selected from the group consisting of hydrogen; alkyl, cycloalkyl, alkenyl, acyl, alkoxy, alkylthio, alkylsulfoxy, alkylsulfonyl, alkylamino, dialkylamino, dialkylphosphinyl, dialkylphosphinoxy and trialkylsilyl radicals having up to 6 carbon atoms; and benzyl, benzoyl, benzyloxy, benzylthio, benzylsulfoxy, benzylsulfonyl, benzylamino, benzoylamido, phenyl, phenoxy, phenylthio, phenylsulfoxy, phenylsulfonyl, phenylamino, phenylacetamido, xylyl, pyridyl and furanyl radicals.

In one embodiment, n is 0 and R is selected from hydrogen, C₁₋₆ alkyl, C₃₋₆ cycloalkyl, phenyl and benzyl radicals.

Illustratively, the phosphonate-containing compound is a homopolymer wherein A in formula (I) is (X)ₙ(R)ₘ where n is 0, m is 1 and R is a C₁₋₆ alkyl or phenyl group, and A′ is hydrogen. Where R is methyl, such a homopolymer is poly(1-phosphonopropene) or a salt thereof. Alternatively, such a homopolymer where R is phenyl is poly(β-styrenephosphonic acid) or a salt thereof.

The phosphonate-containing compound can be present in its phosphonic acid form, where M and M′ are each hydrogen, or as a salt (including partial salt) thereof, wherein, in at least one monomer, at least one of M and M′ is alkali metal, typically sodium or potassium, or ammonium.

In one embodiment the phosphonate-containing compound is a homopolymer of vinylphosphonic acid, or a salt (including partial salt) thereof. Such a compound is described herein as a “polyvinylphosphonate” and can be prepared by any process known in the art, including processes disclosed in above-cited patents and publications.

Whether the phosphonate-containing compound is a polyvinylphosphonate or otherwise, it has an average molecular weight of at least about 1,000, typically about 1,000 to about 100,000 but optionally greater. In various embodiments the average molecular weight of the phosphonate-containing compound is about 5,000 to about 100,000, about 10,000 to about 100,000, about 15,000 to about 100,000, about 20,000 to about 100,000, about 25,000 to about 100,000 or about 25,000 to about 90,000. In one embodiment the average molecular weight is not less than about 22,000, for example about 22,000 to about 90,000, about 22,000 to about 70,000 or about 25,000 to about 35,000. In another embodiment the average molecular weight is not greater than about 20,000, for example about 5,000 to about 20,000 or about 5,000 to about 15,000. It will be noted that for a given polymer or copolymer, number average molecular weights are typically lower than the weight average molecular weights recited herein; for instance a polyvinylphosphonic acid having a weight average molecular weight (MW_{w}) of about 28,000 can have a number average molecular weight (MWₙ) of about 18,000.

As indicated above, the phosphonate-containing compound can be applied to the dental surface in the form of an oral care composition comprising the compound in an orally acceptable vehicle. A suitable amount of the phosphonate-containing compound present in the composition depends on such factors as the particular compound selected, other materials optionally present in the composition, the precise procedure by which contact with the dental surface is effected (e.g., rinsing, brushing, placement of a strip, or chewing) and the desired degree and/or duration of inhibition of staining. Illustratively, whether the phosphonate-containing compound is a polyvinylphosphonate or otherwise, it is usefully present in the composition at a concentration of about 0.1% to about 10% by weight, although greater or lesser concentrations can be useful in particular cases. In one embodiment, the composition comprises a polyvinylphosphonate at about 0.5% to about 5% by weight, for example about 1% to about 4% by weight. Although phosphonate-containing compounds such as polyvinylphosphonic acid (PVPA) can be supplied as dispersions in water, amounts and concentrations are expressed herein on a dry matter (i.e., water-free) basis unless otherwise stated. Also unless otherwise stated, amounts and concentrations of polyvinylphosphonate salts are expressed herein on a PVPA equivalent basis.

An oral care composition comprising a polyvinylphosphonate (PVPA or an alkali metal or ammonium salt thereof) having an average molecular weight of at least about 22,000, for example about 22,000 to about 90,000, in an orally acceptable vehicle, wherein the polyvinylphosphonate is present in an amount effective to inhibit chemical staining of a dental surface, is itself an embodiment of the present invention. Illustrative amounts of the polyphosphonate in the composition are about 0.1% to about 10% by weight, for example about 0.5% to about 5% by weight, or about 1% to about 4% by weight. The polyvinylphosphonate in such an embodiment illustratively has an average molecular weight of about 22,000 to about 70,000, for example about 25,000 to about 35,000.

A composition useful in practicing the method of the present invention can be, for example, a mouthwash, a spray, a dentifrice, an oral strip, a liquid whitener, a chewing gum, a bead, a chew or a lozenge. Dentifrices include without limitation toothpastes, gels and powders. A “liquid whitener” herein encompasses semi-liquid compositions such as gels as well as flowable liquids, so long as the composition is capable of application to a dental surface by painting with a brush or other suitable device. “Painting” herein means application of a thin layer of the composition to the dental surface, as is directed, for example, on the packaging of Colgate® Simply White® Night clear whitening gel sold by Colgate-Palmolive Co., New York, N.Y.

The orally acceptable vehicle of a composition useful according to the invention can comprise any oral care active(s) and/or carrier(s) known in the art. Classification herein of an ingredient as an active or a carrier ingredient is made for clarity and convenience, and no inference should be drawn that a particular ingredient other than the phosphonate-containing compound necessarily functions in the composition in accordance with its classification herein.

Among useful oral care actives are those addressing, without limitation, appearance and structural changes to teeth, treatment and prevention of plaque, calculus, dental caries, cavities, abscesses, inflamed and/or bleeding gums, gingivitis, oral infective and/or inflammatory conditions in general, tooth sensitivity, halitosis and the like. Thus, among useful actives for optional inclusion in a composition useful according to the invention are whitening agents, anticalculus agents, fluoride ion sources, stannous ion sources, zinc ion sources, antimicrobial agents, antioxidants, sialagogues, breath freshening agents, antiplaque agents, anti-inflammatory agents, desensitizing agents, analgesics and nutrients. One active, or more than one active of the same or different classes, can optionally be present. Actives should be selected for compatibility with each other and with other ingredients of the composition.

In one embodiment the composition comprises, in addition to a phosphonate-containing compound, at least one whitening agent. Any orally acceptable whitening agent can be used, including without limitation peroxy compounds, chlorine dioxide, chlorites and hypochlorites. For example, chlorites and hypochlorites of alkali and alkaline earth metals such as lithium, potassium, sodium, magnesium, calcium and barium can be used. Alternatively or in addition, one or more peroxy compounds can be used. Peroxy compounds include hydrogen peroxide, peroxides of alkali and alkaline earth metals, organic peroxy compounds and peroxy acids and salts thereof. Any orally acceptable compound that delivers a perhydroxy (—OOH⁻) ion is useful.

Peroxides of alkali and alkaline earth metals include lithium peroxide, potassium peroxide, sodium peroxide, magnesium peroxide, calcium peroxide and barium peroxide.

Organic peroxy compounds include, for example, carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, monoperoxyphthalate and the like.

Peroxy acids and their salts include organic peroxy acids such as alkyl peroxy acids and monoperoxyphthalate, as well as inorganic peroxy acid salts including persulfate, dipersulfate, percarbonate, perphosphate, perborate and persilicate salts of alkali and alkaline earth metals such as lithium, potassium, sodium, magnesium, calcium and barium.

One or more whitening agents are optionally present in a tooth-whitening effective total amount, typically about 0.1% to about 90%, for example about 0.5% to about 50% or about 1% to about 30% by weight of the composition. Where peroxy compounds such as hydrogen peroxide are included, they can suitably be present in a total hydrogen peroxide equivalent amount of about 0.5% to about 50%, for example about 1% to about 30% by weight of the composition. Peroxy compounds can illustratively be present in a total hydrogen peroxide equivalent amount of about 2% to about 10% by weight in a dentifrice composition, or about 10% to about 30% by weight in a liquid whitener composition.

In one embodiment a composition is provided, comprising in an orally acceptable vehicle (a) at least one whitening agent in a total amount effective to partially or completely remove an existing chemical stain from a dental surface, and (b) at least one phosphonate-containing compound as defined herein, for example a polyvinylphosphonate of average molecular weight about 4,000 to about 90,000, in a total amount effective to inhibit chemical staining due to exposure of the surface to a chemical stain inducing agent after application of the composition to the surface.

A dentifrice illustrative of a particular embodiment can comprise, for example, about 0.5% to about 5% by weight PVPA and about 1% to about 10% by weight hydrogen peroxide.

A liquid whitener illustrative of another particular embodiment can comprise, for example, about 0.5% to about 5% by weight PVPA and about 10% to about 30% by weight hydrogen peroxide.

In a further embodiment a composition useful according to the invention comprises, in addition to a phosphonate-containing compound, at least one anticalculus agent. Any orally acceptable anticalculus agent can be used, including without limitation phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts illustratively include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and the like, wherein sodium can optionally be replaced by potassium or ammonium. Other useful anticalculus agents include polycarboxylate polymers and polyvinyl methyl ether/maleic anhydride (PVME/MA) copolymers, such as those available under the Gantrez™ brand from ISP, Wayne, N.J. One or more anticalculus agents are optionally present in an anticalculus effective total amount, typically about 0.01% to about 50%, for example about 0.05% to about 25% or about 0.1% to about 15% by weight of the composition.

In a still further embodiment a composition useful according to the invention comprises, in addition to a phosphonate-containing compound, at least one fluoride ion source useful, for example, as an anti-caries agent. Any orally acceptable fluoride ion source can be used, including without limitation potassium, sodium and ammonium fluorides and monofluorophosphates, stannous fluoride, indium fluoride and the like. Water-soluble fluoride ion sources are typically used. One or more fluoride ion sources are optionally present in an amount providing a total of about 0.0025% to about 2%, for example about 0.005% to about 1% or about 0.01% to about 0.3%, of fluoride ions by weight of the composition.

In a still further embodiment a composition useful according to the invention comprises, in addition to a phosphonate-containing compound, at least one stannous ion source useful, for example, in helping reduce gingivitis, plaque, caries or sensitivity. Any orally acceptable stannous ion source can be used, including without limitation stannous fluoride, other stannous halides such as stannous chloride dihydrate, organic stannous carboxylate salts such as stannous formate, acetate, gluconate, lactate, tartrate, oxalate, malonate and citrate, stannous ethylene glyoxide and the like. One or more stannous ion sources are optionally and illustratively present in a total amount of about 0.01% to about 10%, for example about 0.1% to about 7% or about 1% to about 5% by weight of the composition.

The composition can optionally comprise at least one zinc ion source useful, for example, as an antimicrobial, anticalculus or breath-freshening agent. Any orally acceptable zinc ion source can be used, including without limitation zinc citrate, zinc sulfate, zinc glycinate, sodium zinc citrate and the like. One or more zinc ion sources are optionally and illustratively present in a total amount of about 0.05% to about 3%, for example about 0.1% to about 1%, by weight of the composition.

In a still further embodiment a composition useful according to the invention comprises, in addition to a phosphonate-containing compound, at least one antimicrobial (e.g., antibacterial) agent. Any orally acceptable antimicrobial agent can be used, including without limitation triclosan (5-chloro-2-(2,4-dichlorophenoxy)phenol), 8-hydroxyquinoline and salts thereof, copper (II) compounds such as copper (II) chloride, fluoride, sulfate and hydroxide, phthalic acid and salts thereof such as magnesium monopotassium phthalate, chlorhexidine, alexidine, hexetidine, sanguinarine, benzalkonium chloride, salicylanilide, domiphen bromide, alkylpyridinium chlorides such as cetylpyridinium chloride (CPC) (including combinations of CPC with zinc and/or enzymes), tetradecylpyridinium chloride and N-tetradecyl-4-ethylpyridinium chloride, octenidine, iodine, sulfonamides, bisbiguanides, phenolics, piperidino derivatives such as delmopinol and octapinol, zinc ion sources, magnolia extract, grapeseed extract, phenol, thymol, eugenol, menthol, geraniol, carvacrol, citral, eucalyptol, catechol, 4-allylcatechol, hexyl resorcinol, 2,2′-methylene bis(4-chloro-6-bromophenol), methyl salicylate, antibiotics such as augmentin, amoxicillin, tetracycline, doxycycline, minocycline, metronidazole, neomycin, kanamycin and clindamycin, and the like. A further illustrative list of useful antibacterial agents is provided in above-cited U.S. Pat. No. 5,776,435. One or more antimicrobial agents are optionally present in an antimicrobial effective total amount.

In a still further embodiment a composition useful according to the invention comprises, in addition to a phosphonate-containing compound, at least one antioxidant. Any orally acceptable antioxidant can be used, including without limitation butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin and the like. One or more antioxidants are optionally present in an antioxidant effective total amount.

In a still further embodiment a composition useful according to the invention comprises, in addition to a phosphonate-containing compound, a sialagogue (saliva stimulating agent), useful for example in amelioration of dry mouth. Any orally acceptable sialagogue can be used, including without limitation food acids such as citric, lactic, malic, succinic, ascorbic, adipic, fumaric and tartaric acids. One or more sialagogues are optionally present in the composition in a saliva stimulating effective total amount.

In a still further embodiment a composition useful according to the invention comprises, in addition to a phosphonate-containing compound, a breath freshening agent. Any orally acceptable breath freshening agent can be used, including without limitation zinc salts such as zinc gluconate, zinc citrate and zinc chlorite, α-ionone and the like. One or more breath freshening agents are optionally present in a breath freshening effective total amount.

In a still further embodiment a composition useful according to the invention comprises, in addition to a phosphonate-containing compound, an antiplaque, including plaque disrupting, agent. Any orally acceptable antiplaque agent can be used, including without limitation stannous, copper, magnesium and strontium salts, dimethicone copolyols such as cetyl dimethicone copolyol, papain, glucoamylase and glucose oxidase. One or more antiplaque agents are optionally present in an antiplaque effective total amount.

In a still further embodiment a composition useful according to the invention comprises, in addition to a phosphonate-containing compound, at least one anti-inflammatory agent. Any orally acceptable anti-inflammatory agent can be used, including without limitation steroidal agents such as flucinolone and hydrocortisone, and nonsteroidal agents (NSAIDs) such as ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone and phenylbutazone. One or more anti-inflammatory agents are optionally present in an anti-inflammatory effective amount.

In a still further embodiment a composition useful according to the invention comprises, in addition to a phosphonate-containing compound, at least one desensitizing agent. Potassium salts such as potassium nitrate are illustratively useful in this regard, as is sodium nitrate. Alternatively or in addition a local or systemic analgesic such as aspirin, codeine, acetaminophen, sodium salicylate or triethanolamine salicylate can be used. One or more desensitizing agents and/or analgesics are optionally present in a desensitizing and/or analgesic effective amount.

In a still further embodiment a composition useful according to the invention comprises, in addition to a phosphonate-containing compound, at least one nutrient. Suitable nutrients include vitamins, minerals and amino acids.

Among useful carriers for optional inclusion in a composition useful according to the invention are diluents, abrasives, bicarbonate salts, pH modifying agents, surfactants, foam modulators, thickening agents, viscosity modifiers, humectants, sweeteners, flavorants and colorants. One carrier material, or more than one carrier material of the same or different classes, can optionally be present. Carriers should be selected for compatibility with each other and with other ingredients of the composition.

Water is a preferred diluent and in some compositions such as mouthwashes and whitening liquids is commonly accompanied by an alcohol, e.g., ethanol. The weight ratio of water to alcohol in a mouthwash composition is generally about 1:1 to about 20:1, for example about 3:1 to about 20:1 or about 4:1 to about 10:1. In a whitening liquid, the weight ratio of water to alcohol can be within or below the above ranges, for example about 1:10 to about 2:1.

In one embodiment a composition useful according to the invention comprises, in addition to a phosphonate-containing compound, at least one abrasive, useful for example as a polishing agent. Any orally acceptable abrasive can be used, but type, fineness (particle size) and amount of abrasive should be selected so that tooth enamel is not excessively abraded in normal use of the composition. Suitable abrasives include without limitation silica, for example in the form of silica gel, hydrated silica or precipitated silica, alumina, insoluble phosphates, calcium carbonate, resinous abrasives such as urea-formaldehyde condensation products and the like. Among insoluble phosphates useful as abrasives are orthophosphates, polymetaphosphates and pyrophosphates. Illustrative examples are dicalcium orthophosphate dihydrate, calcium pyrophosphate, β-calcium pyrophosphate, tricalcium phosphate, calcium polymetaphosphate and insoluble sodium polymetaphosphate. One or more abrasives are optionally present in an abrasive effective total amount, typically about 5% to about 70%, for example about 10% to about 50% or about 15% to about 30% by weight of the composition. Average particle size of an abrasive, if present, is generally about 0.1 to about 30 μm, for example about 1 to about 20 μm or about 5 to about 15 μm.

In a further embodiment a composition useful according to the invention comprises, in addition to the phosphonate-containing compound, at least one bicarbonate salt, useful for example to impart a “clean feel” to teeth and gums due to effervescence and release of carbon dioxide. Any orally acceptable bicarbonate can be used, including without limitation alkali metal bicarbonates such as sodium and potassium bicarbonates, ammonium bicarbonate and the like. One or more bicarbonate salts are optionally present in a total amount of 0.1% to about 50%, for example about 1% to about 20% by weight of the composition.

In a still further embodiment a composition useful according to the invention comprises, in addition to the phosphonate-containing compound, at least one pH modifying agent. Such agents include acidifying agents to lower pH, basifying agents to raise pH and buffering agents to control pH within a desired range. For example, one or more compounds selected from acidifying, basifying and buffering agents can be included to provide a pH of about 2 to about 10, or in various illustrative embodiments about 2 to about 8, about 3 to about 9, about 4 to about 8, about 5 to about 7, about 6 to about 10, about 7 to about 9, etc. Any orally acceptable pH modifying agent can be used, including without limitation carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole and the like. One or more pH modifying agents are optionally present in a total amount effective to maintain the composition in an orally acceptable pH range.

In a still further embodiment a composition useful according to the invention comprises, in addition to the phosphonate-containing compound, at least one surfactant, useful for example to compatibilize other components of the composition and thereby provide enhanced stability, to help in cleaning the dental surface through detergency, and to provide foam upon agitation, e.g., during brushing with a dentifrice composition of the invention. Any orally acceptable surfactant, most of which are anionic, nonionic or amphoteric, can be used. Suitable anionic surfactants include without limitation water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable nonionic surfactants include without limitation poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants include without limitation derivatives of C₈₋₂₀ aliphatic secondary and tertiary amines having an anionic group such as carboxylate, sulfate, sulfonate, phosphate or phosphonate. A suitable example is cocoamidopropyl betaine. One or more surfactants are optionally present in a total amount of about 0.01% to about 10%, for example about 0.05% to about 5% or about 0.1% to about 2% by weight of the composition.

In a still further embodiment a composition useful according to the invention comprises, in addition to the phosphonate-containing compound, at least one foam modulator, useful for example to increase amount, thickness or stability of foam generated by the composition upon agitation. Any orally acceptable foam modulator can be used, including without limitation polyethylene glycols (PEGs), also known as polyoxyethylenes. High molecular weight PEGs are suitable, including those having an average molecular weight of about 200,000 to about 7,000,000, for example about 500,000 to about 5,000,000 or about 1,000,000 to about 2,500,000. One or more PEGs are optionally present in a total amount of about 0.1% to about 10%, for example about 0.2% to about 5% or about 0.25% to about 2% by weight of the composition.

In a still further embodiment a composition useful according to the invention comprises, in addition to the phosphonate-containing compound, at least one thickening agent, useful for example to impart a desired consistency and/or mouth feel to the composition. Any orally acceptable thickening agent can be used, including without limitation carbomers, also known as carboxyvinyl polymers, carrageenans, also known as Irish moss and more particularly t-carrageenan (iota-carrageenan), cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, colloidal silica and the like. One or more thickening agents are optionally present in a total amount of about 0.01% to about 15%, for example about 0.1% to about 10% or about 0.2% to about 5% by weight of the composition.

In a still further embodiment a composition useful according to the invention comprises, in addition to the phosphonate-containing compound, at least one viscosity modifier, useful for example to inhibit settling or separation of ingredients or to promote redispersibility upon agitation of a liquid composition. Any orally acceptable viscosity modifier can be used, including without limitation mineral oil, petrolatum, clays and organomodified clays, silica and the like. One or more viscosity modifiers are optionally present in a total amount of about 0.01% to about 10%, for example about 0.1% to about 5% by weight of the composition.

In a still further embodiment a composition useful according to the invention comprises, in addition to the phosphonate-containing compound, at least one humectant, useful for example to prevent hardening of a toothpaste upon exposure to air. Any orally acceptable humectant can be used, including without limitation polyhydric alcohols such as glycerin, sorbitol, xylitol or low molecular weight PEGs. Most humectants also function as sweeteners. One or more humectants are optionally present in a total amount of about 1% to about 50%, for example about 2% to about 25% or about 5% to about 15% by weight of the composition.

In a still further embodiment a composition useful according to the invention comprises, in addition to the phosphonate-containing compound, at least one sweetener, useful for example to enhance taste of the composition. Any orally acceptable natural or artificial sweetener can be used, including without limitation dextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, cyclamates and the like. One or more sweeteners are optionally present in a total amount depending strongly on the particular sweetener(s) selected, but typically about 0.005% to about 5% by weight of the composition.

In a still further embodiment a composition useful according to the invention comprises, in addition to the phosphonate-containing compound, at least one flavorant, useful for example to enhance taste of the composition. Any orally acceptable natural or synthetic flavorant can be used, including without limitation vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or warming effects. Such ingredients illustratively include menthol, menthyl acetate, menthyl lactate, camphor, eucalyptus oil, eucalyptol, anethole, eugenol, cassia, oxanone, α-irisone, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of about 0.01% to about 5%, for example about 0.1% to about 2.5% by weight of the composition.

In a still further embodiment a composition useful according to the invention comprises, in addition to the phosphonate-containing compound, at least one colorant. Colorants herein include pigments, dyes, lakes and agents imparting a particular luster or reflectivity such as pearling agents. A colorant can serve a number of functions, including for example to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including without limitation talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride and the like. One or more colorants are optionally present in a total amount of about 0.001% to about 20%, for example about 0.01% to about 10% or about 0.1% to about 5% by weight of the composition.

Degree of staining or stain inhibition on a dental surface can be observed visually, for example with the aid of color comparison charts, gauges or shade guides, e.g., as described by Browning (2003), Journal of Esthetic Restorative Dentistry 15 Supp. 1, S13-S20, incorporated herein by reference.

Alternatively, staining or inhibition thereof can be measured by colorimetry, using any suitable instrument such as a Minolta Chromameter, e.g., model CR-321 (Minolta Corp., Ramsey, N.J.). The instrument can be programmed, for example, to measure Hunter Lab values or L*a*b* values according to the standard established by the International Committee of Illumination (CIE). The L*a*b* system provides a numerical representation of three-dimensional color space where L* represents a lightness axis, a* represents a red-green axis and b* represents a yellow-blue axis. The L* and b* axes are typically of greatest applicability to tooth stain inhibition, which can be measured as increase in whiteness relative to an untreated surface. Increase in whiteness can be computed from differences in L*, a* and b* values between untreated and treated surfaces. A useful parameter is ΔE*, calculated as the square root of the sum of the squares of differences in L*, a* and b* values, using the formula:
ΔE*=[(ΔL*)²+(Δa*)²+(Δb*)²]^{1/2}
A higher value of ΔE* indicates greater increase in whiteness.

Evaluation of effectiveness of stain inhibition treatments of the invention can be made, for example, in clinical studies using human volunteers, or in vivo in animals, conducted according to appropriate protocols.

Suitable in vitro protocols are also available for evaluation of stain inhibition treatments, including those described in Examples herein and in published literature. See for example Stookey et al. (1982), Journal of Dental Research 61(11), 1236-1239, and Rice et al. (2001), Journal of Clinical Dentistry 12(2), 34-37, both incorporated herein by reference.

The invention can further be understood by reference to the following nonlimiting examples.

### EXAMPLES

### Example 1

A toothpaste was prepared having the composition shown in Table 1. The glycerin, carboxymethylcellulose sodium and 1-carrageenan were mixed together for at least about 5 minutes. The sorbitol, water, titanium dioxide, sodium saccharin and sodium fluoride were then added and the resulting mixture was heated to 60-71° C. with mixing for at least about 15 minutes. The PVPA and sodium hydroxide were then added with mixing for at least about 5 minutes. The hydrated silica was then added and mixing continued for at least about 15 minutes under vacuum. Finally, the sodium lauryl sulfate powder and flavorant were added and mixing continued under vacuum for at least a further 10 minutes.

TABLE 1
Composition of toothpaste of Example 1

| Ingredient | Weight % |
|---|---|
| water | 16.11 |
| sodium fluoride | 0.24 |
| sodium saccharin | 0.30 |
| glycerin | 20.00 |
| CMC sodium | 1.10 |
| ι-carrageenan | 0.40 |
| sorbitol | 25.78 |
| titanium dioxide | 0.50 |
| polyvinylphosphonic acid (PVPA), 32% in water | 9.38 |
| sodium hydroxide | 2.20 |
| hydrated silica | 21.50 |
| flavorant | 1.00 |
| sodium lauryl sulfate powder | 1.50 |

The PVPA used in this example was supplied by Clariant Corp., Charlotte, N.C. and had a weight average molecular weight of 28,000 and a number average molecular weight of 18,000.

Effectiveness of the composition of Example 1 in inhibition of staining of a dental surface, relative to water and a placebo toothpaste (having composition similar to that of Example 1 but lacking PVPA) was determined by the following procedure, adapted from Baig et al. (2002), op. cit.
1. Human saliva, kept on ice until needed, was centrifuged at 10,000 rpm for 10 minutes at room temperature. The supernatant was collected and kept on ice until needed.
2. Disks of synthetic hydroxyapatite (SHAP, to simulate a natural dental surface) were rinsed in water, blotted and allowed to air-dry. Their lightness of color L* as established by CIE was measured using a Minolta CR-321 chromameter.
3. The SHAP disks were then placed in a 17×100 mm polystyrene test tube, one disk per tube, and 2 ml of saliva supernatant was added to each disk. The test tubes were incubated in a shaker bath at 37° C. overnight.
4. The disks were removed from the saliva supernatant, rinsed in water and blotted dry, and were then returned to the test tubes.
5. A slurry was prepared of the toothpaste composition at a 1:10 dilution in water, and 2 ml of the slurry was added to each disk, followed by incubation in the shaker bath at 37° C. for 5 minutes.
6. The disks were removed from the toothpaste slurry, rinsed in water and blotted dry, and were then returned to the test tubes.
7. A staining material (coffee beverage prepared from Flavia® Arabica Full Roast coffee blend at a 1:10 dilution in water) was added to each disk, followed by incubation in the shaker bath at 37° C. for 15 minutes. Alternative staining materials that have been used in similar tests include (1) instant coffee, dissolved in boiling water, and (2) a 0.12% chlorhexidine rinse (Periogard®) followed by (1).
8. The disks were removed from the staining material, rinsed in water and blotted dry, and were then returned to the test tubes.
9. A further 2 ml of saliva supernatant was added to each disk, followed by incubation in the shaker bath at 37° C. for 20 minutes.
10. The disks were removed from the saliva supernatant, rinsed in water and blotted dry, and were then returned to the test tubes.
11. Steps 7-10 were repeated to provide a total of three cycles of exposure to the staining material.
12. A further 2 ml of the toothpaste slurry was added to each disk, followed by incubation in the shaker bath at 37° C. for 5 minutes.
13. The disks were rinsed in water, blotted dry and allowed to air dry. A further measurement of L* was obtained.
14. Inhibition of staining was determined as ΔL*, the difference in L* between disks treated with the toothpaste of Example 1 and those treated with water or the placebo toothpaste.

The toothpaste composition of Example 1 exhibited ΔL* of 63% versus water and 58% versus the placebo toothpaste. This result demonstrates a high degree of effectiveness of polyvinylphosphonate as a toothpaste ingredient in inhibiting staining of dental surfaces.

### Example 2

A liquid whitener was prepared having the composition shown in Table 2.

TABLE 2
Composition of liquid whitener of Example 2

| Ingredient | Weight % |
|---|---|
| water | 6.25 |
| carbomer | 1.00 |
| PEG, average MW 600 | 10.00 |
| PEG, average MW 2,000,000 | 14.00 |
| butylated hydroxytoluene | 0.03 |
| glycerin | 5.00 |
| polyvinylphosphonic acid (PVPA), 32% in water | 3.13 |
| sodium hydroxide | 0.75 |
| ethanol, 95% | 34.80 |
| hydrogen peroxide | 25.00 |
| monosodium phosphate | 0.05 |

### Example 3

A liquid whitener was prepared having the composition shown in Table 3.

TABLE 3
Composition of liquid whitener of Example 3

| Ingredient | Weight % |
|---|---|
| water | 21.74 |
| carbomer | 1.00 |
| PEG, average MW 600 | 5.00 |
| PEG, average MW 2,000,000 | 15.00 |
| butylated hydroxytoluene | 0.03 |
| polyvinylphosphonic acid (PVPA), 32% in water | 3.13 |
| phosphoric acid, 85% | 0.10 |
| ethanol, 95% | 35.00 |
| hydrogen peroxide | 18.00 |
| monosodium phosphate | 0.05 |
| trisodium phosphate | 1.00 |

The PVPA used in Examples 2 and 3 was the same as that used in Example 1.

Effectiveness of the composition of Example 3 in inhibition of staining of a dental surface, relative to water and a placebo liquid whitener (having composition similar to that of Example 3 but lacking PVPA) was determined by a procedure similar to that described in Example 1. The liquid whitener composition of Example 3 exhibited ΔL* of 66% versus water and 44% versus the placebo liquid whitener. This result demonstrates a high degree of effectiveness of polyvinylphosphonate as a liquid whitener ingredient in inhibiting staining of dental surfaces.

1. A method for inhibiting chemical staining of a dental surface, the method comprising contacting the dental surface with an orally acceptable polymer or copolymer that comprises a plurality of monomeric groups having the formula 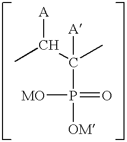
wherein (a) one of A and A′ is hydrogen and the other is a moiety (X)ₙ(R)ₘ, (b) n in individual such moieties is independently 0 or 1, (c) linking groups X if present independently comprise an oxygen, sulfur, nitrogen, phosphorus or silicon atom, (d) where n is 0, m is 1, and where n is 1, m is independently an integer from 1 to 3 as determined by X, (e) terminal groups R are independently hydrogen or C₁₋₁₈ organic radicals, and (f) M and M′ are independently selected from hydrogen, alkali metal and ammonium; said polymer or copolymer having an average molecular weight of at least about 1,000.

2. The method of claim 1 wherein, after contacting the dental surface with the polymer or copolymer, the dental surface is exposed to a chemical stain inducing material, and wherein chemical staining resulting from such exposure is inhibited by said contacting with the polymer or copolymer.

3. The method of claim 2 wherein the chemical stain inducing material is selected from the group consisting of tobacco products, tea and coffee.

4. The method of claim 1 wherein the dental surface is contacted with an oral care composition that comprises said polymer or copolymer in an amount effective to inhibit chemical staining of the dental surface, in an orally acceptable vehicle.

5. The method of claim 4 wherein the contacting of the dental surface with the oral care composition is effected by a procedure that comprises rinsing, spraying, brushing or painting the surface with the composition, placement of the composition on the surface or chewing the composition.

6. The method of claim 4 wherein said polymer or copolymer has an average molecular weight of about 1,000 to about 100,000.

7. The method of claim 4 wherein said polymer or copolymer has an average molecular weight of about 5,000 to about 100,000.

8. The method of claim 4 wherein said polymer or copolymer has an average molecular weight of about 10,000 to about 100,000.

9. The method of claim 4 wherein, in said polymer or copolymer, the monomeric groups are recurring groups.

10. The method of claim 9 wherein, in said monomeric groups, said (X)ₙ(R)ₘ moiety is selected from the group consisting of hydrogen; alkyl, cycloalkyl, alkenyl, acyl, alkoxy, alkylthio, alkylsulfoxy, alkylsulfonyl, alkylamino, dialkylamino, dialkylphosphinyl, dialkylphosphinoxy and trialkylsilyl radicals having up to 6 carbon atoms; and benzyl, benzoyl, benzyloxy, benzylthio, benzylsulfoxy, benzylsulfonyl, benzylamino, benzoylamido, phenyl, phenoxy, phenylthio, phenylsulfoxy, phenylsulfonyl, phenylamino, phenylacetamido, xylyl, pyridyl and furanyl radicals.

11. The method of claim 9 wherein, in said monomeric groups, n is 0, m is 1 and R is selected from hydrogen, C₁₋₆ alkyl, C₃₋₆ cycloalkyl, phenyl and benzyl radicals.

12. The method of claim 9 wherein, in said monomeric groups, A is said moiety (X)ₙ(R)ₘ and A′ is hydrogen.

13. The method of claim 9 wherein said polymer or copolymer is a polyvinylphosphonate that comprises recurring monomeric groups having the formula 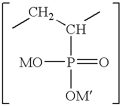
where M and M′ are each hydrogen, alkali metal or ammonium and are the same or different, said polyvinylphosphonate having an average molecular weight of about 1,000 to about 100,000.

14. The method of claim 13 wherein the composition comprises said polyvinylphosphonate in a polyvinylphosphonic acid equivalent amount of about 0.1% to about 10% by weight.

15. The method of claim 13 wherein the composition comprises said polyvinylphosphonate in a polyvinylphosphonic acid equivalent amount of about 0.5% to about 5% by weight.

16. The method of claim 13 wherein said orally acceptable vehicle comprises at least one ingredient selected from the group consisting of whitening agents, anticalculus agents, fluoride ion sources, stannous ion sources, zinc ion sources, antimicrobial agents, antioxidants, saliva stimulating agents, breath freshening agents, antiplaque agents, anti-inflammatory agents, desensitizing agents, analgesics, nutrients, diluents, abrasives, bicarbonate salts, pH modifying agents, surfactants, foam modulators, thickening agents, viscosity modifiers, humectants, sweeteners, flavorants and colorants.

17. The method of claim 13 wherein said orally acceptable vehicle comprises a whitening agent selected from the group consisting of peroxy compounds, chlorites, hypochlorites and combinations thereof.

18. The method of claim 17 wherein said whitening agent comprises a peroxy compound.

19. The method of claim 18 wherein said peroxy compound is selected from the group consisting of hydrogen peroxide, peroxy organic compounds and peroxy acids.

20. The method of claim 13 wherein said composition is selected from the group consisting of mouthwashes, sprays, dentifrices, oral strips, liquid whiteners, chewing gums, beads, chews and lozenges.

21. The method of claim 13 wherein said composition is a dentifrice and wherein contacting of the dental surface is effected by brushing the surface with the composition.

22. The method of claim 13 wherein said composition is a liquid whitener and wherein contacting of the dental surface is effected by painting the surface with the composition.

23. The method of claim 13 wherein said polyvinylphosphonate has an average molecular weight of about 22,000 to about 90,000.

24. An oral care composition comprising, in an orally acceptable vehicle, an amount, effective to inhibit chemical staining of a dental surface, of a polyvinylphosphonate that comprises recurring monomeric groups having the formula 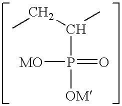
where M and M′ are each hydrogen, alkali metal or ammonium and are the same or different, said polyvinylphosphonate having an average molecular weight of about 22,000 to about 90,000.

25. The composition of claim 24 that comprises said polyvinylphosphonate in a polyvinylphosphonic acid equivalent amount of about 0.1% to about 10% by weight.

26. The composition of claim 24 that comprises said polyvinylphosphonate in a polyvinylphosphonic acid equivalent amount of about 0.5% to about 5% by weight.

27. The composition of claim 24 wherein said orally acceptable vehicle comprises at least one ingredient selected from the group consisting of whitening agents, anticalculus agents, fluoride ion sources, stannous ion sources, antimicrobial agents, antioxidants, saliva stimulating agents, breath freshening agents, antiplaque agents, anti-inflammatory agents, desensitizing agents, analgesics, nutrients, diluents, abrasives, bicarbonate salts, pH modifying agents, surfactants, foam modulators, thickening agents, viscosity modifiers, humectants, sweeteners, flavorants and colorants.

28. The composition of claim 24 that is selected from the group consisting of mouthwashes, dentifrices, oral strips, liquid whiteners and chewing gums.

29. An oral care composition comprising, in an orally acceptable vehicle, (a) at least one whitening agent in a total amount effective to partially or completely remove an existing chemical stain from a dental surface and (b) a polyvinylphosphonate that comprises recurring monomeric groups having the formula 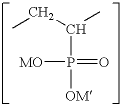
where M and M′ are each hydrogen, alkali metal or ammonium and are the same or different, said polyvinylphosphonate having an average molecular weight of about 4,000 to about 90,000, in a total amount effective to inhibit chemical staining due to exposure of the surface to a chemical stain inducing agent after application of the composition to the surface.

30. The composition of claim 29 that comprises said polyvinylphosphonate in a polyvinylphosphonic acid equivalent amount of about 0.1% to about 10% by weight.

31. The composition of claim 29 that comprises said polyvinylphosphonate in a polyvinylphosphonic acid equivalent amount of about 0.5% to about 5% by weight.

32. The composition of claim 29 wherein the whitening agent is selected from the group consisting of peroxy compounds, chlorites, hypochlorites and combinations thereof.

33. The composition of claim 32 wherein said whitening agent comprises a peroxy compound.

34. The composition of claim 33 wherein said peroxy compound is selected from the group consisting of hydrogen peroxide, peroxy organic compounds and peroxy acids.

35. The composition of claim 29 that is selected from the group consisting of mouthwashes, sprays, dentifrices, oral strips, liquid whiteners, chewing gums, beads, chews and lozenges.

36. The composition of claim 29 that is a dentifrice comprising about 1% to about 10% by weight hydrogen peroxide and about 0.5% to about 5% by weight polyvinylphosphonic acid.

37. The composition of claim 29 that is a liquid whitener comprising about 10% to about 30% by weight hydrogen peroxide and about 0.5% to about 5% by weight polyvinylphosphonic acid.
